(19) United States
(12) Patent Application Publication
(10) Pub. No.: US 2006/0034878 A1
(43) Pub. Date: Feb. 16, 2006

(54) Iodized oil

(76) Inventor: Artus L. Satterfield, Irvington, NJ (US)

Correspondence Address: Artus L. Satterfield, 221 19th Ave., IRVINGTON, NJ, 07111, US

(21) Appl. No.: 10/919,456

(22) Filed: Aug. 16, 2004

(51) Int. Cl. A61K 31/225; A61K 8/37

(52) U.S. Cl. 424/401; 514/547

(57) ABSTRACT

Microorganism resistant heavy and light oils with suspended resublimed iodine for dermal protection.

### BACKGROUND—DESCRIPTION OF PRIOR ART

Tincture of iodine (iodine in alcohol) stains the skin.

### OBJECT AND ADVANTAGE

Iodized oil does not stain the skin. This is important for face and other exposed skin application.

Resublimed iodine is suspended in light (mineral oil) and or heavy (petrolatum) oils.

### DESCRIPTION

Heavy oil (petralatum) is melted over low heat and removed from heat.

Resublimed iodine is added to melted oil and dispensed and allowed to solidify.

Thin oils do not require heating.

### APPLICATION

Heavy or light iodized oil is applied to skin and scalp to control microorganisms.

### SUMMARY, RAMIFICATIONS AND SCOPE

Iodized oil is a stainless microorganism resistant oil that is suitable for hair care and dermal cosmetics.

1. Heavy and light microorganism resistant iodized oil for dermal protection.
